(19) United States
(12) Patent Application Publication
(10) Pub. No.: US 2026/0283457 A1
(43) Pub. Date: Sep. 24, 2026

(54) AN EAR PROBE FOR EAR EXAMINATION

(71) Applicant: Otos Health Oy, Helsinki (FI)

(72) Inventors: Wycliffe Raduma, Helsinki (FI); Saku Sinkkonen, Helsinki (FI); Ville Sivonen, Helsinki (FI); Tanja Tervonen, Helsinki (FI)

(73) Assignee: Otos Health Oy, Helsinki (FI)

(21) Appl. No.: 19/480,086

(22) PCT Filed: May 8, 2024
(86) PCT No.: PCT/FI2024/050222

§ 371 Date: Oct. 30, 2025

(30) Foreign Application Priority Data
May 8, 2023 (FI) 20237087

(51) Int. Cl. A61B 1/227; A61B 1/00; A61B 1/04; A61B 1/07; A61B 5/00; A61B 5/01; A61B 5/107; A61B 5/12; A61B 7/02; H04N 23/50; H04N 23/55; H04N 23/58; H04R 1/10

(52) U.S. Cl. CPC A61B 1/227; A61B 1/00032; A61B 1/041; A61B 1/07; A61B 5/01; A61B 5/1076; A61B 5/1079; A61B 5/126; A61B 5/6817; A61B 7/023; H04N 23/55; H04N 23/555; H04N 23/58; H04R 1/1008; H04R 1/1083; H04R 2460/01

(57) ABSTRACT

There is provided an ear probe for ear examination. The ear probe includes a waveguide with an elongated tube, wherein the waveguide is configured to transfer light. A capsule is fixed to the waveguide and includes an image sensor. The image sensor is configured to generate multiple images of an ear canal and/or tympanic membrane. The waveguide is configured to guide the capsule to an ear canal of an ear to be examined, and the waveguide is configured to adapt to structure of an ear to be examined.

### TECHNICAL FIELD

The present invention relates to an ear probe for ear examination, a headset comprising the ear probe, uses of the ear probe, and a measurement device for processing data of the ear probe.

### BACKGROUND

The need for hearing care in the future is huge. WHO (2021) has estimated that 2.5 billion people will have some degree of hearing loss by 2050, and 1/14 of them would need hearing rehabilitation. For example, in Europe some degree of hearing loss has been predicted to increase up to 236 million people by 2050. The cost of hearing loss is estimated at $981 billion worldwide. Therefore, early identification of hearing loss and ear diseases is the key to effective treatment (WHO 2021). Not only hearing loss causes costs to society but there is also ample evidence linking Age-Related Hearing Impairment (ARHI) and changes in cognitive ability. Therefore, prevention efforts in the form of hearing aid rehabilitation should be considered to activate the aging brain and increase socialization.

### SUMMARY

Aim is to enable an optical measurement of an ear.

The invention is defined by the features of the independent claims. Some embodiments are defined in the dependent claims.

According to a first aspect of the present invention, there is provided an ear probe for ear examination comprising a waveguide comprising an elongated tube, wherein the waveguide is configured to transfer light, and a capsule fixed to the waveguide and including an image sensor. The image sensor configured to generate multiple images of an ear canal and/or tympanic membrane. The waveguide is configured to guide the capsule to an ear canal of an ear to be examined, and the waveguide is configured to adapt to structure of an ear to be examined.

According to a second aspect of the present invention, there is provided a headset comprising the ear probe according to first aspect.

According to a third aspect of the present invention, there is provided a measurement device comprising a processor and a memory configured to store data and executable instructions that when executed by the processor, are configured to cause the measurement device at least to: receive data measured by the ear probe according to the first aspect; generate a data structure based on the received data; convert the data structure to a desired form; pack the converted data into exportable form; and transmit the converted data structure in the exportable form.

According to a fourth aspect of the present invention, there is provided uses of the ear probe according to the first aspect, for performing simultaneously imaging of the ear canal and/or tympanic membrane, and a hearing test; for acoustic reflectometry; for performing for tympanometry; for recording otoacoustic emissions and/or for performing noise cancellation.

### BRIEF DESCRIPTION OF THE DRAWINGS

In the following embodiments are discussed in more detail with reference to the attached drawings, of which:

FIG. 1A illustrates, by way of an example, an ear probe for examining ears.

FIG. 1B illustrates, by way of an example, an ear probe for examining ears with a headset.

FIG. 1C illustrates, by way of an example, an ear probe for examining ears with a headset.

FIG. 2 illustrates, by way of an example, an ear probe for examining ears.

FIG. 3 illustrates, by way of an example, an ear probe for examining ears.

FIG. 4 illustrates, by way of an example, an ear probe for examining ears.

FIG. 5 illustrates, by way of an example, a measurement device for ear probe data.

FIG. 6 illustrates, by way of an example, a measurement device for ear probe data.

Figures are presented as illustrative examples and embodiments may not be limited solely to the illustrated parts, but modifications may be made under the scope as defined in the claims. Figures that may not fully present the claimed invention, aim to provide better understanding on the context and relating technical field.

### DESCRIPTION OF EMBODIMENTS

There is provided an ear probe for examining ears, for example, a middle ear, an ear canal and/or a tympanic membrane (eardrum). The ear probe comprises a waveguide, a capsule at an end of the waveguide, and an optical sensor, or an image sensor. The waveguide may comprise a tube or cable. The waveguide is configured to transfer data, sound, light and/or power. An optical element may be an imaging-or a camera sensor at the capsule of the waveguide. An optical element is configured to generate images of the examined ear, like middle ear, ear canal and/or tympanic membrane. The waveguide is flexible, such that the waveguide is configured to conform to a shape of an ear canal and an outer ear.

The ear probe is configured to perform otoscopy. Otoscopy is enabled by optics which is situated at one end of a waveguide. The waveguide may comprise an elongated tube, or alike pipe like structure, having a hollow inside for transferring e.g., light, sound and/or power.

An otoscope or an auriscope is a medical device that a doctor or other medical workers use to look into a patient's ear. An otoscope is a tool which, typically enlarges and straightens an ear canal, and shines a beam of light in order to help visualize and examine an ear canal and an eardrum (i.e. tympanic membrane). Otoscopy is a clinical procedure used to examine structures of the ear, like auditory canal, tympanic membrane and middle ear.

The capsule houses an image sensor. The image sensor or a camera sensor is integrated to the capsule. The capsule is configured to be inserted to an ear to be examined, with aid of a waveguide. The waveguide is configured to adapt, conform and/or accommodate shapes of the ear to be examined, without exerting excessive force on the ear or any part of it, like ear canal, tympanic membrane and/or possible ear wax. Flexibility or adaptability of the waveguide enables to conform with any ear canal. Further, it allows the capsule to be placed in the ear canal and fit around possible obstacles. This is enabled by carrying, directing, guiding and/or manoeuvring the capsule around possible obstacles, like ear wax. A waveguide, which is configured to carry the capsule, may have limited amount of force that it is able to exert on an obstacle in the ear canal, for example on tympanic membrane. Such makes the ear probe safe to use. In addition, the capsule may comprise an element configured to measure orientation of the capsule. The element may comprise a gyroscope, a micro-electromechanical (MEMS) gyroscope, an acceleration sensor, a mechanical element, or any alike element capable of measuring angular motion or orientation. With aid of the element the capsule may provide feedback of its current orientation. The feedback may be live feedback via live video feed (of the camera of the capsule) and/or other positional telemetry. Data of the element configured to measure orientation may be used to orientate images of the image sensor (of the capsule). This way correctly oriented image is received, despite the actual orientation of the ear probe.

In addition, other optoacoustic sensors may be attached or connected to the ear probe; or be part of or integrated to the ear probe. In addition to an image sensor, optoacoustic sensor may comprise a microphone, a speaker, a reflectometer, a temperature sensor, an infrared (IR) sensor, or alike optoacoustic sensor. An ear probe for ear examination, or a capsule of it, may comprise an element configured to orientate the capsule, the image sensor, and/or images from the image sensor. The element may comprise a gyroscope, a MEMS gyroscope, an acceleration sensor, a circuit, or any other element capable for orienting the capsule, the image sensor (camera) and/or images of the image sensor (camera) of the capsule. The acoustic elements may placed outside the ear canal. For example, sound may be produced by a speaker placed outside an ear canal, and directed to the ear canal. The ear canal otoacoustic emissions, or resonance, caused in response to a known sound produced by the speaker outside of the ear canal, may be measured using a microphone. A microphone picks up the sound. A microphone may be placed or integrated in the capsule. A microphone may be attached to a tube configured to carry sound to a microphone, which may be placed next to the ear probe, next to the waveguide, next to the capsule, or outside of the ear canal. The microphone does not have to be physically on the ear probe, but it may be connected to a tube that carries the sound to a microphone.

Any additional sensors used with the ear probe may be integrated to the ear probe and/or to the capsule of it. Alternatively or in addition, any additional sensors may external to the ear probe, and/or connectable to the ear probe. Any additional sensors may be provided via the waveguide of the ear probe; or via a tube or a waveguide of their own, which may be placed next to, or inside the waveguide of the ear probe.

An ear probe comprises a waveguide and a capsule to be inserted to an ear canal using the waveguide. The waveguide is flexible. Flexibility is ability of the waveguide, or material of it, to deform elastically and to return to its original shape, when any applied stress to it is removed. The waveguide is configured to bend and flex in order to be used in and near ear, or for ear examination. The waveguide may comprise a tube of one or more of: plastic, thermoplastic, moulded plastic, elastomer, soft elastomer, and/or silicon. Many synthetic elastomers and plastics have strength and flexibility. The material of the waveguide may be such that it withstands sterilization and/or cleaning. Waveguide may be made of silicon, or any other suitable material used for the medical products.

An ear probe may be called an optoacoustic ear probe, in case an optical element and an acoustic element have been combined or integrated to the ear probe. The embodiments enable a combination of an otoscope and an audiometry apparatus in one device. An optical element, or an image sensor, is configured to image and an acoustic element is configured to measure otoacoustic emissions. An optoacoustic ear probe enables otoscopy and audiometry to be performed by a single device, or an ear probe. Data for both may be recorded simultaneously. No user activity is required.

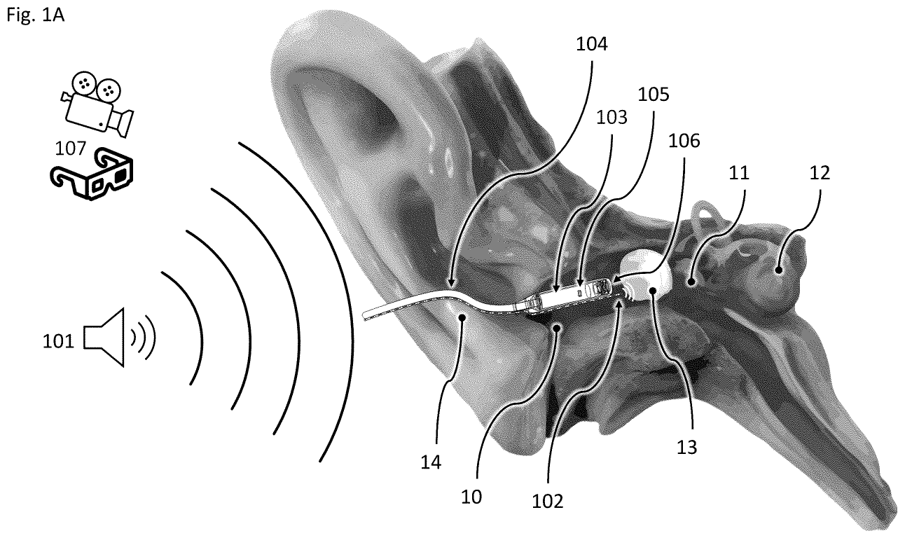

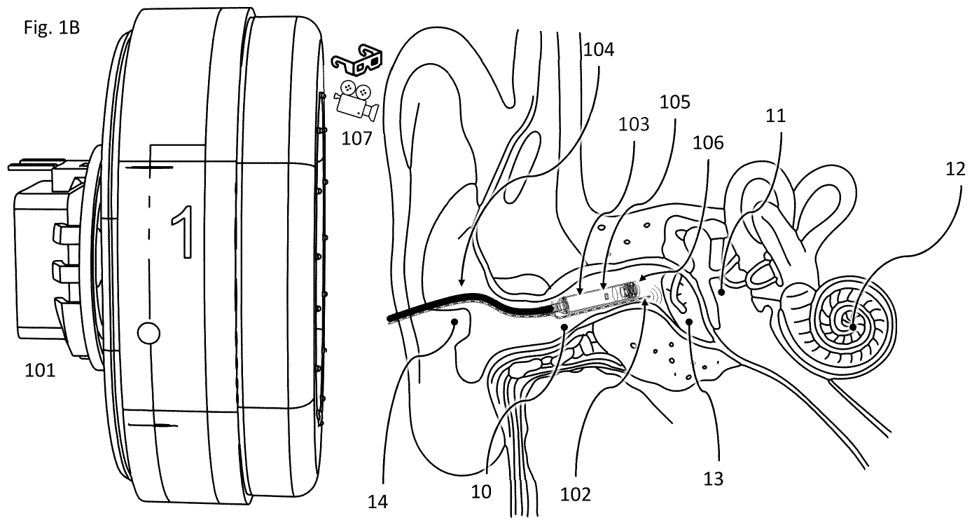

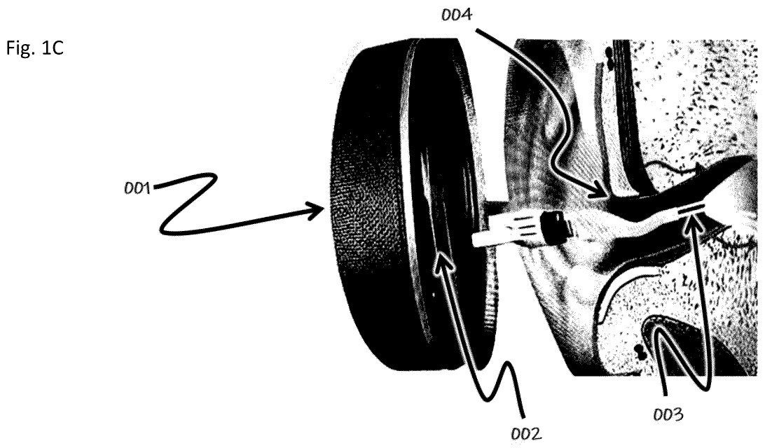

FIG. 1A illustrates, by way of an example. an ear probe for examining ears. FIG. 1 illustrates an outer ear 14, for example curving around the tragus, ear canal 10, middle ear 11, and a tympanic membrane 10. A ear probe comprising a waveguide 104 and a capsule 103 at an end of the waveguide 104 is illustrated in an ear canal 10. As FIG. 1 shows, the waveguide 104 enables insertion of the ear probe to different kind of ears. Earlier otoscopes are known to be solid and non-flexible such that those actually have enlarged and straightened an ear canal from outer ear towards the tympanic membrane. The ear probe of FIG. 1 enables more user-friendly examination of ears of different users, which may be more suitable for kids and people with disabilities.

The waveguide 104 is structured for data, sound, light and/or power transfer. An illumination element 106 is arranged to provide light within the ear, like ear canal 10. An illumination element 106 may be integrated in the capsule 103. In alternative, or additionally, an illumination element 106 may be at the end or other part of the capsule 103, or external to the capsule, or the ear probe; and configured to direct light to the ear, at the end of the capsule 103. The capsule 103 is arranged at an end of the waveguide 104. The capsule 103 is at an end of the waveguide 104, which is inserted to an ear to be examined. The capsule 103 comprises an optical sensor 106, like an image sensor, a camera sensor, or alike. The optical sensor 106 is placed at the capsule 103. The optical sensor 106 is integrated in or at the capsule 103. In FIG. 1 the image sensor is arranged the end of the capsule 103, which is opposite to the end of the capsule 103 attached to the waveguide 104. The image sensor enables to use the ear probe for otoscopy. Additional image sensors or camera sensors 107 may be place for example outside of the ear canal 10. Supplemental images of the additional imaging-/camera sensors 107 may aid or improve in production of three-dimensional images and/or scans.

The ear probe or the capsule 103 may have a detachably attachable or an integrated acoustic element 102. The acoustic element 102 may enter to the ear canal 10 with the ear probe or the capsule 103. The acoustic element 102 is configured to detect, record and/or pick up sound or otoacoustic emissions. Sound may be produced by external acoustic element 101 outside the ear canal 10; or in the ear canal 10 by an acoustic element, which may be built in the capsule; or by the acoustic element 102. The acoustic element 102 may comprise a receiver and a transmitter, like a microphone and a speaker.

The capsule 103 may comprise an element 105 configured to detect orientation of the capsule 103. Orientation of the capsule 103 enables easier use of the ear probe. Detected orientation may be informed to a user of the ear probe, or used for re-orienting the images of the optical sensor 106 of the capsule 103. The capsule 103 may be cleanable, for single-use, or protectable, for example via cleanable and/or disposable covers. Cleanable and/or disposable covers may be configurable with acoustic elements 105 in the ear canal 10.

FIG. 1B illustrates, by way of an example, an ear probe for examining ears. FIG. 1B uses the same numbering for similar part with FIG. 1A. A waveguide 104 is configured to guide a capsule 103 to an ear canal 10, via outer ear 14. The ear probe comprises an image sensor 102. In addition, the ear probe may comprise an element configured to detect orientation 105, an illumination element 106, external optical sensors 107, and or other sensors, like a temperature sensor. The ear probe of FIG. 1B may be attached to an earcup 101 of an earphone. The earphone may be used as an acoustic element, for example a sound source. The waveguide may be fixed to the earcup of the earphone. The wave guide may be flexible, or flexibly attached to the earcup 101 of the earphone.

FIG. 1C illustrates, by way of an example, an ear probe for examining ears. The ear probe comprises a capsule 003, an image sensor and an acoustic element. The ear probe may comprise a mirror and/or a lens system, like a two lens system. The mirror and/or the lens system enables the image sensor to capture, at once, two or more images having different viewing angles. Thus, an examined ear canal may be visualized more accurately or widely at once, compared to a single image of an image sensor of the capsule. The mirror and/or lens system creates different images from different viewing angles, which support the photogrammetric reconstruction of a three-dimensional (3D) image. In addition or alternatively, the images may be used to provide additional, computer-generated focal lengths, in order to provide images with more points on focus, or other computer-aided optical reconstruction.

A capsule comprises an image sensor. The image sensor may comprise a camera sensor, a camera, or alike suitable for providing a digital otoscope or a three-dimensional (3D) scan.

In addition to one or more 3D image, it is possible to combine pure tone audiometry for testing individual sounds with the ear probe, which may be working as an otoscope or a 3D otoscope. Testing individual sounds enables providing a quick overview of patient's hearing level, for example in the absence of a complete audiogram.

A real ear measurement (REM) apparatus may be attached to or integrated into an ear probe as an acoustic element. Attached or integrated REM enables to perform a hearing test at the same time with otoscopy. The hearing test may be based on causing and measuring optoacoustic emissions in the ear canal and/or tympanic membrane. A speaker (a loudspeaker) may be configured to emit an acoustic emission to an ear canal. A microphone may be configured to receive acoustic emission or resonance from the ear canal. An external speaker may be utilized. In alternative an acoustic element may comprise receiver-transceiver, for example a microphone and a speaker. Otoacoustic emission in an ear canal may be measured. An ear canal is known to strengthen some frequencies and to reduce other frequencies of otoacoustic emission. REM, audiometric testing may be performed during otoscopy and without patient involvement. REM is a passive objective measurement.

An audiogram may be built using Pure Tone Audiometry. Otoacoustic emission (OAE) is an objective way to create an audiogram, which comprises hearing thresholds. In case an EEG is further used, auditory brain response (ABR) is another objective way to produce an audiogram.

An acoustic element may be a microphone, a microphone-speaker. In addition, further acoustic elements may be used, like a microphone, a speaker, a linear actuator, or alike acoustic element; for example, controlled extreme offset of the speaker element. A linear actuator may be used for causing movement of a tympanic membrane via pressure. An ear probe working as an otoscope or a 3D otoscope may comprise a built-in linear actuator as an acoustic element. A linear actuator may be used to direct low frequencies, like inaudible low frequencies, to an ear. Driving low frequencies with high amplitude causes movement of a tympanic membrane. This replaces traditional otoscope by acting as a substitute for a manually operated pump of the traditional otoscope.

An ear probe comprises two opposing ends. One of the ends is fixed to the waveguide, and the other, opposing end is a trailing end for entering to the ear, or ear canal. The latter is called an examination end. A microphone may be at the examination end of the capsule, which is opposing the end of the capsule fixed to the waveguide. A waveguide may comprise a capsule at one end, and optionally a handle at its opposing end. A microphone may receive sound and/or direct sound via the probe, to the end of the probe configured to enter to an ear canal. This enables for example REM testing to be performed. In addition or alternative, a microphone may be inside of the capsule. A microphone may be at a circuit board or printed circuit board, PCB, which is in the capsule.

In FIG. 1C, a headphone 001 is used as an acoustic element of an ear probe. The acoustic element can be an existing certified and calibrated audiometry headphone or a purpose-built headphone for this solution. An image sensor for otoscopy and an acoustic element for audiometry can be a retrofitted addon that attaches to the inside of the audiometer speaker 002 while preserving acoustic properties and calibration levels of the headphone 001. The addon elements may replace a cushion of the headphone 001 earcup, at least partly. The addon element may be placed between the headphone 001 earcup and an ear of a user of the headphone 001. The airways 004 in the ear canal, around the hearing probe are not limiting the delivery on intended acoustic signal and pressure in a significant way. In case a pneumatic test is to be used, the airways 004 shall be closed. This may be implemented by using an earcup of a headset for closing the airways around the hearing probe 004. In case of an ear probe without a headset or earcup, sealing of airways of ear canal may be done by introducing a foam to the ear canal, or by a waveguide structure, which is able to enlarge in order to seal the ear canal. This is useful for example, when pressure of a tympanic membrane is measured.

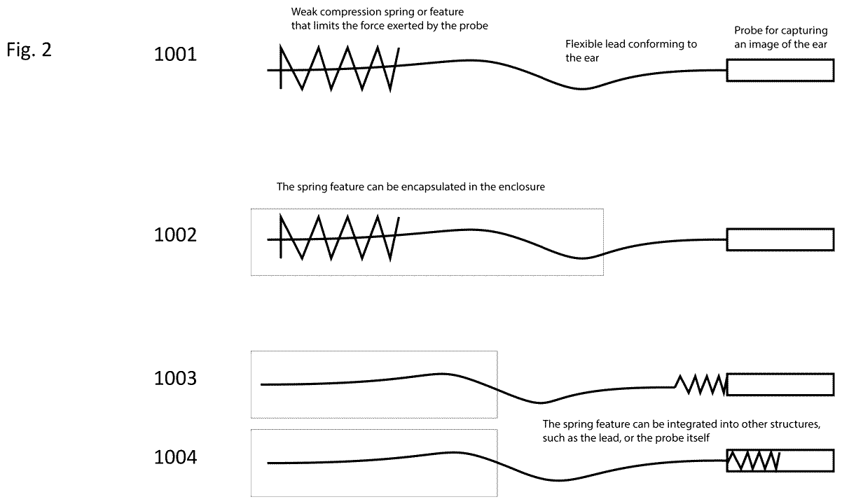

FIG. 2 illustrates, by way of an example, an ear probe. An ear probe may be made safe by providing force-limiting functionality. As discussed in the previous, an ear probe comprises conformity to adapt to an ear to be examined. Such conformity, or flexibility may be implemented via physical properties of an ear probe. A right side of FIG. 2 illustrates a capsule, which is configured enter to an ear canal. The capsule comprises an image sensor for capturing one or more images. Examination ends of the capsules are at the right end of the FIG. 2. The capsules are connected to a waveguide at their end opposing the examination end. In order to provide force-limiting functionality that limits a force exerted by an ear probe, a spring is introduced, as an example, in the ear probes of FIG. 2. A compression spring of 1001 may be a weak compression spring. In alternative, other feature that is configured to limit a force exerted by the ear probe may be utilized in any of the examples of FIG. 2. A compression spring of 1002 is encapsulated in an enclosure, which may be a handle of the ear probe. A spring may be integrated into other structures, like spring 1003 is integrated to a waveguide, or spring 1004 is integrated to the capsule of the ear probe. Spring

An ear probe is inserted to an ear of a patient. The hearing probe comprises an optical element and an acoustic element integrated to the hearing probe. In addition, other complementary diagnostic tools may be integrated to the hearing probe. An image of an ear canal and a tympanic membrane may be achieved by one or more image sensor, and possible optical element(s), of the ear probe. This way one or more optical element enables the ear probe to be used as an otoscope. Acoustic measurements, such as screening pure tone audiometry (for determining or inspecting hearing thresholds for certain frequencies that can be plotted on an audiogram), real ear measurements (REM), Tympanometry, or otoacoustic emissions (OAE), may be achieved by an acoustic element of the ear probe. The one or more acoustic elements enable the ear probe to be used for getting data for acoustic measurements. An electroencephalogram (EEG) EEG is a test that measures electrical activity in the brain using small electrodes. For EEG readings, electrodes are attached for example to a headset comprising the hearing probe. An Open Brain Computer Interface (OpenBCI), being a tool to sample an electrical activity, may be combined with an EEG sensor and the ear probe for measuring EEG. A graph measured via a reflectometer. A reflectometer enables to measure an inflammation of an ear. Measured results may be viewed by a graph showing clear difference for a detected inflammation of an ear, and a healthy ear. With an integrated reflectometer, the optoacoustic hearing probe may be as an acoustic reflectometer.

In addition to an image sensor, and possible acoustic elements, other functional elements may be added or combined with the ear probe. An acoustic reflectometer may be combined with or integrated to an ear probe. A temperature measurement may be combined with or integrated to an ear probe. Integration refers to a fixed placement to an ear probe, for example to a capsule. Combination may be implemented inside the waveguide and/or capsule, or next to the ear probe, and/or externally to the ear probe, via a tube of sensor's own, or via waveguide of the ear probe.

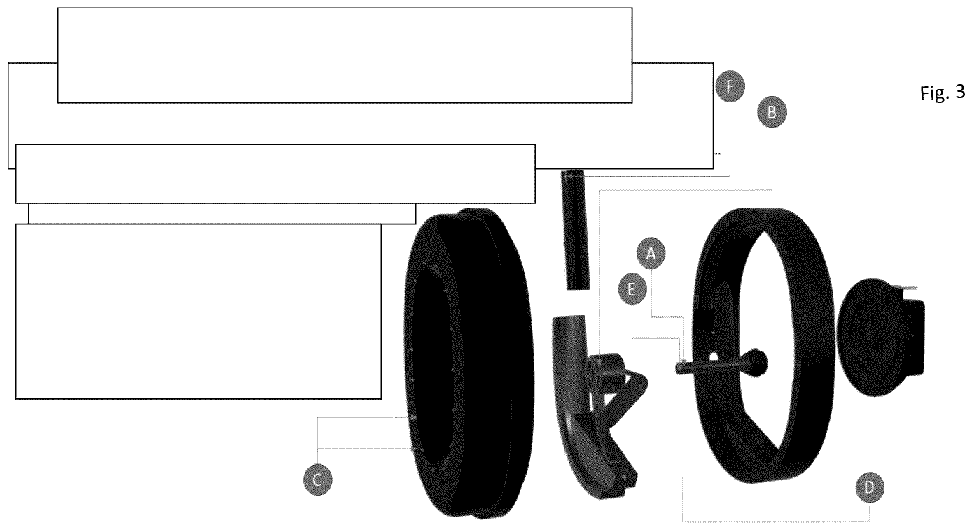

FIG. 3 illustrates, by way of an example, an ear probe. FIG. 3 (A) illustrates a camera module integrated to an ear probe. A waveguide may act as a semi-stiff string that will bend and give, if accidentally pushed until the eardrum or into an ear canal-obstructing object. This aids in placement of the hearing probe to the ear canal. A temperature sensor may be integrated to the ear probe. Temperature measurement may be added to an imaging or camera sensor. The temperature sensor may comprise an infrared (IR) sensor. Since the optoacoustic hearing probe may be kept in the ear canal for a while, a thermocouple, which may be IP rated, may be used for temperature measurement.

FIG. 3 (B) illustrates an integrated acoustic element, being a microphone in FIG. 3 (B). The microphone may be used for detecting and/or recording otoacoustic emissions. Otoacoustic emissions (OAE) are sounds generated form the cochlea transmitted across the middle ear to the external ear canal, where they may be recorded using the microphone. OAE recordings enable an audiogram to be created. The audiogram may include hearing thresholds.

An acoustic reflectometry typically involves propagating an audio signal at a number of frequencies through the ear canal.

A mentioned in the previous, an acoustic reflectometry, a tympanometry and/or a noise cancellation may be performed. The microphone may be used to detect an ear probe noise signal, which may be used for noise cancellation, for example for a headset.

Tympanometry measures how an eardrum moves. For accurate measurements, ear canal should be sealed without leaving free airways for the measurements. The microphone may receive and enable measuring resonance or optoacoustic emission from the ear canal. A reflected frequencies from the ear canal may be recorded and analysed in order to determine mechanical resonance characteristics of the tympanic membrane.

FIG. 3 (C) illustrates an EEG element, or cushion, at least partly around an earcup of a headset. The EEG element may comprise pogo pins, so called spring-loaded pins as dry (gel free) electrodes. The electrodes with suitable detected contact and signal may be selected for use in data acquisition. An EEG element is an optional element of the hearing probe.

FIG. 3 (D) illustrates pneumatics for detecting eardrum movement, tympanometry or improved acoustic sealing of the optical (camera) probe of FIG. 3. Also, barometric pressure sensors may be used for pressure-regulation feedback loops.

FIG. 3 (E) illustrates an image sensor integrated to the hearing probe. The image sensor element comprises optics for photogrammetric or stereoscopic 3D imaging. One or more additional optical element(s) may comprise an image sensor, a camera sensor, a mirror and/or two lenses, for example.

FIG. 3 (F) illustrates module comprising a battery and a circuit. The circuit comprises a logic unit for the hearing probe. The battery and the circuit are replaceable and/or upgradable. The module (F) may be changed or replaced. This enables easier and cheaper service and upgrades for the hearing probe. This reduces product risk.

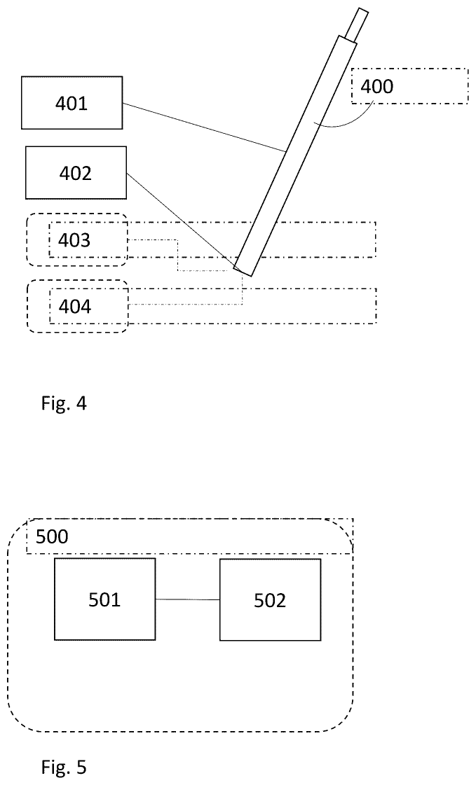

FIG. 4 illustrates, by way of an example, an ear probe 400. An image sensor is 401 is integrated to the ear probe 400. In addition, an acoustic element 402, like a microphone or a microphone-speaker, may be combined or integrated to the ear probe. Further, other elements 403 or sensors may be combined or integrated to the ear probe 400. Other elements 403 may comprise one or more of the following: a reflectometer, a temperature sensor, an acoustic sensor, a microphone, a speaker, a microphone-speaker. A measurement device 404 may be connected to and/or a part of to the ear probe 400.

FIG. 5 illustrates, by way of an example, a measurement device. Measurement device 500 comprises a processor 501 and a memory 502. The memory 502 is configured to store data, for example data measured by the ear probe, as discussed in the previous. The memory 502 may comprise executable instructions for processing data generated by the ear probe, upon executing the executable instructions by the processor 501. The measurement device 500 includes a processor 501 and a memory 502 in FIG. 5.

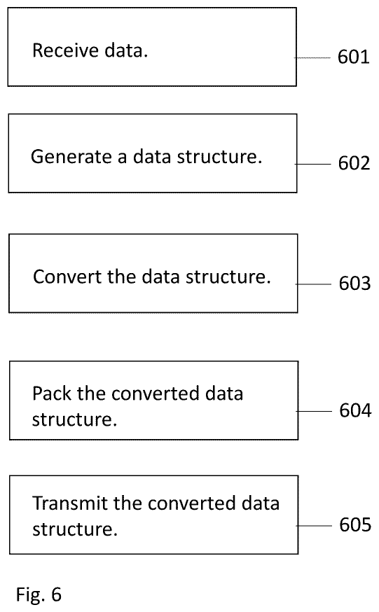

FIG. 6 illustrates, by way of an example, a measurement device. The measurement device, e.g., of FIG. 5, is be caused to receive data, at a phase 601, measured by an ear probe. The measurement device is caused to generate a data structure, at a phase 602, based on the received data. The data structure, like a file and/or a folder, which may include metadata on an examined patient, and so on. The measurement device is caused to convert the data structure 603 to a desired form. The form may be compatible with Health Level 7 and/or DICOM standard, or any other existing a future form. A standardized form enables to utilize data without further conversions. Medical devices are often DICOM compatible, in which case no additional adapters are needed. Also, Health Level 7, HL7, compatible files are widely used. Therefore, no external conversions are needed, but the conversion may be implemented at the measurement device. The measurement device is caused to pack the converted data 604 into an exportable form. Packed data may be exported to a desired receiver device. Further, the measurement device may be caused to transmit the converted data structure 605 in the exportable form. The measurement device may be caused to transmit data wirelessly or via cable.

An ear probe, or a headset comprising a hearing probe, or alike device, may be connected to, and/or co-operate with a measurement device. Edge computing may be utilized to produce preliminary/suggested results on the device, without a need for an active internet connection. A measurement device may be placed at a controller of an ear probe, or at a handle of an ear probe.

The measurement device may comprise a server. The measurement device may comprise a component for conversion of data into a desired form, e.g., HL7 or DICOM. The measurement device is capable of packaging data into exportable file formats, and/or transmitting the data wirelessly or via cables. Additional processing of the results can be done remotely or using online platforms or tools.

The described ear probe, having a flexible waveguide for guiding the capsule to an ear, enables use of the ear probe for different kinds and sizes of ears. The described ear probe is capable of performing and acquiring an otoscopic image of an ear, like an ear canal and tympanic membrane. In addition, a passive hearing test, e.g., a Pure Tone Audiometry, may be implemented. A hearing test and otoscopic imaging may be combined to a single system, like to a single optoacoustic ear probe, to a headset, or alike device. The embodiments enable to evolve an imaging ear probe with added features, for example a microphone configured to record otoacoustic emissions, which enables performing an objective hearing test. A passive hearing test does not require patient activity. This enables to make objective hearing tests, without patient involvement. This is useful for example, for testing kids or disabled persons.

The described hearing probe may enable to provide detailed information for automated artificial intelligence (AI)-based analysis and diagnosis.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

The described features, structures, or characteristics may be combined in any suitable manner in one or more embodiments. In the previous description, numerous specific details are provided, such as examples of structures, lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

1. An ear probe for ear examination comprising:
a waveguide comprising an elongated tube, wherein the waveguide is configured to transfer light,
a capsule fixed to the waveguide and including an image sensor,
the image sensor configured to generate multiple images of an ear canal and/or tympanic membrane,
the waveguide is configured to guide the capsule to an ear canal of an ear to be examined, and
the waveguide is configured to adapt to structure of an ear to be examined.

2. An ear probe according to the claim 1, comprising an acoustic element configured to record otoacoustic emission.

3. (canceled)

4. An ear probe according to claim 1, wherein the capsule is fixed at an end of the waveguide.

5. (canceled)

6. (canceled)

7. An ear probe according to claim 1, further comprising a mirror system and/or lenses.

8. An ear probe according to claim 7, wherein the image sensor is configured to create images of an ear canal and/or tympanic membrane with the mirror system and/or lenses from different viewing angles.

9. An ear probe according to claim 8, wherein the created images are configured to reconstruct a three-dimensional image of the ear canal and/or tympanic membrane; to generate a computer-aided optical reconstruction of the ear canal and/or tympanic membrane; and/or to generate multi-focal length images of the ear canal and/or tympanic membrane.

10. (canceled)

11. (canceled)

12. An ear probe according to claim 1, comprising at least one further acoustic element integrated to, attached to, or combined with, the ear probe.

13. An ear probe according to the claim 12, wherein the at least one the further acoustic element comprises a loudspeaker, a speaker, a microphone or a linear actuator.

14. An ear probe according to the claim 13, wherein at least one of: the loudspeaker, the speaker and/or the linear actuator is configured to direct low frequencies to the ear canal.

15. (canceled)

16. An ear probe according to claim 1, further comprising one or more additional acoustic sensors and/or imaging sensors.

17. An ear probe according to claim 1, further comprising an acoustic reflectometer integrated to, attached to, or combined with, the ear probe.

18. An ear probe according to claim 1, further comprising a temperature sensor integrated to or combined with or attached to the ear probe.

19. An ear probe according to claim 1, further comprising an external speaker for performing a real ear measurement, wherein the external speaker is configured to produce sound to an ear canal to be examined, in order to provide microphone-detectable otoacoustic emission in the ear canal.

20. (canceled)

21. An ear probe according to claim 1, wherein the waveguide is expandable in order to fill the ear canal.

22. A headset comprising the ear probe according to claim 1.

23. (canceled)

24. A headset according to claim 22, further comprising an integrated circuit and a battery for ear probe, wherein the integrated circuit and the battery are replaceable.

25. A measurement device comprising a processor and a memory configured to store data and executable instructions that when executed by the processor, are configured to cause the measurement device at least to:
receive data measured by the ear probe according to claim 1;
generate a data structure based on the received data,
convert the data structure to a desired form;
pack the converted data into exportable form; and
transmit the converted data structure in the exportable form.

26. The measurement device according to the claim 24, which is further caused at least to convert the data structure in a form of Health Level 7 or DICOM standard.

27. (canceled)

28. The measurement device according to claim 24, which is further caused to transmit the converted data structure in the exportable form wirelessly or via cable.

29. A use of the ear probe according to claim 2 for performing simultaneously imaging of the ear canal and/or tympanic membrane, and a hearing test.

30. A use of the ear probe according to claim 17 for acoustic reflectometry.

31. (canceled)

32. (canceled)

33. A use of the ear probe according to claim 2 for performing noise cancellation.
